(19) United States
(12) Patent Application Publication
(10) Pub. No.: US 2004/0057985 A1
(43) Pub. Date: Mar. 25, 2004

(54) Transdermal therapeutic system comprising the active ingredient oxybutynin

(76) Inventor: Stefan Bracht, Ochtendung (DE)

Correspondence Address: BIRCH STEWART KOLASCH & BIRCH, PO BOX 747, FALLS CHURCH, VA, 22040-0747, US

(21) Appl. No.: 10/433,698

(22) PCT Filed: Nov. 24, 2001
(86) PCT No.: PCT/EP01/13678

(30) Foreign Application Priority Data
Dec. 6, 2000 (DE) 10060550.8

(51) Int. Cl. A61K 9/70; A61K 31/24

(52) U.S. Cl. 424/449; 514/540

(57) ABSTRACT

A transdermal therapeutic system (TTS) for administering the active substance oxybutynin, comprising a substantially water vapour-impermeable backing layer (1), at least one pressure-sensitive adhesive matrix layer (2, 3) attached thereto, and a detachable protective film (4), is characterized in that the said matrix layer comprises two phases which are immiscible with each other, namely an inner and an outer phase, with the said inner phase (2) containing the active substance oxybutynin base or oxybutynin hydrochloride and being dispersed in the form of droplets in the outer phase (3), and the said outer phase being a pressure sensitive adhesive prepared on the basis of hydrocarbon polymers or/and silicone polymers.

The present invention relates to transdermal therapeutic systems (TTS) for the administration of the active substance oxybutynin. The invention further relates to a process of producing oxybutynin-containing active substance layers of transdermal therapeutic systems.

Oxybutynin is an anticholinergic and spasmolytic which is utilized above all for treating disturbed function of the bladder, especially strangury, incontinence, or nycturia. Commonly, this active substance is administered orally as oxybutynin hydrochloride, for example in the form of tablets, capsules or syrup.

Apart from the above, transdermal therapeutic systems have been described in the literature, which are to enable the administration of this active substance via the skin. By way of example, reference is made in this connection to the patent specification of the firm of ALZA (U.S. Pat. No. 5,500,222, U.S. Pat. No. 5,411,740, U.S. Pat. No. 5,900,250 and EP 721 349), of the firm of Theratech (U.S. Pat. No. 5,834,010) and of Schwarz Pharma AG (DE 198 12 413 C1).

The majority of these patent specifications, however, stated the necessity that in order to achieve therapeutically active absorption rates of oxybutynin via the skin, a permeation-enhancing additive (enhancer) must be present in the TTS. The following substances were proposed as enhancers in this context: monoglycerides or fatty acids (U.S. Pat. No. 5,500,222, U.S. Pat. No. 5,411,740), a mixture of monoglycerides and lactate esters (U.S. Pat. No. 5,900,250) or triacetin (U.S. Pat. No. 5,834,010).

The use of enhancers does, however, involve an increased risk of skin irritations occurring. Generally, it is valid that the addition of enhancers should, if possible, be avoided if the required transdermal absorption rates can also be attained without any such additives.

It is true that in DE 198 12 413 C1 it could be shown that the required flow rates could also be achieved with a transdermal system without the addition of an enhancer, but the system assemblies described therein are directed to hot-melt technology. These enhancer-free formulations are prepared on the basis of ammoniogroup-containing (meth)acrylate polymers. The hot-melt method used in this process necessitates the use of plasticizers, in this case plasticizers of the group of the citric acid esters. This constitutes a severe limitation as the clear majority of TTS market products and the production sites existing therefor are geared to the solvent-based production, not to hot-melt technology.

It was therefore the object of the present invention to provide transdermal therapeutic systems for the administration of oxybutynin with which therapeutically active absorption rates can be achieved without the necessity of adding permeation-enhancing substances (enhancers), and which can be produced on the basis of solvent-containing processes in an economical manner and on a large scale, and do not necessitate the use of hot-melt processes.

This object could be solved with the, surprisingly simple, system assembly described in claim 1; further, especially useful, embodiments are described in the subclaims. The inventive TTS, having the features mentioned in the introductory portion of claim 1, are characterized in that they comprise at least one active substance-containing matrix layer which is substantially made up of at least two phases (2, 3) which are immiscible with one another. These are an inner phase and an outer phase, with the inner phase (2) containing the active substance oxybutynin base or oxybutynin hydrochloride and being dispersed in droplet form in the outer phase (3). The outer phase is a pressure sensitive adhesive prepared on the basis of hydrocarbon polymers or/and silicone polymers.

A further administration form provides that in the inner phase, apart from oxybutinine, there is also contained its pharmacodynamically active main metabolite desethyloxybutynin. Preferably, oxybutynin and desethyloxybutinine are present in a weight ratio of from 1:10 to 10:1.

Furthermore, it is preferred for at least 90% of oxybutynin and, if present in the TTS, of desethyloxybutynin as well to be present as (S)-enantiomer.

With preference, the active substance(s) is/are present in dissolved form, with at least 50%-wt. of the active substance being dissolved, especially preferred 90-100%.

Through the inventive matrix assembly of two phases, with the active substance solution or active substance-containing preparation being dispersed or emulsified in droplet form in a surrounding polymer phase, it is possible to make optimum use of the thermodynamic activity of the active substance. As a consequence, it is not necessary to add any enhancer substances to achieve sufficient skin permeation rates.

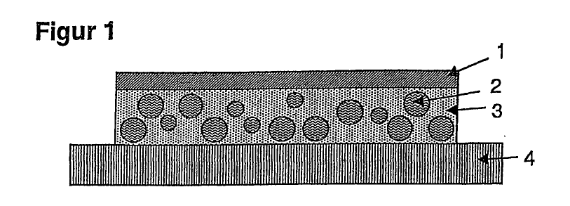

The structure of an inventive transdermal therapeutic system is depicted by way of example in FIG. 1 (sectional representation). The active substance(s) or the active substance solution (possibly in combination with a binder polymer) forms the inner phase (2) and is present dispersed in droplet-form in a surrounding, pressure sensitive adhesive outer phase (3). The system is provided on the side averted from the skin with a preferably water vapour-impermeable backing layer (1), as well as, on the skin-contact side, with a detachable protective layer (4). This exemplary basic type can be modified in various ways, as described hereinbelow. In addition, the TTS can be manufactured in different geometric surface shapes, e.g. round, oval or oblong.

In the simplest case, the inner phase (2) consists exclusively of the liquid active substance solution or dispersion. This corresponds to a droplet-like distribution of the active substance within an outer phase oversatured with the active substance, and ensures maximum thermodynamic activity of the active substance. Especially preferred is, however, an embodiment wherein the active substance-containing inner phase (2) contains an addition of one or more binding agents (also called thickening agents). In this way it can be prevented that the active substance of the droplet-shaped inner phase accumulates at the interfaces and surfaces of the matrix layer, which could affect the adhesive power of the pressure-sensitive adhesive matrix layer. In addition, the active substance solution emerging at the interfaces would act as an abherent, so that these layers could no longer be laminated with films, e.g. PET films, as backing layer (1).

Surprisingly, it was found that this phenomenon can be very effectively suppressed or totally prevented by adding certain polymers as binding or thickening agents to the inner phase (2). Given these conditions it is possible to incorporate at least 5%-wt., preferably 10 to 25%-wt, of the active substance oxybutynin into the matrix without the active substance exudating or emerging at the surface of the active substance matrix.

On the other hand, the polymer should be added to the inner phase (2) in as small an amount as possible, preferably the portion thereof should at the most equal the weight percentage of the oxybutynin contained. An excessively high portion of binding agent polymer in the inner phase could unnecessarily lower the thermodynamic activity of the active substance owing to its solubility in the binding agent. The binding or thickening agent is preferably present in a portion of at least 10%-wt, preferably from 10-50%-wt, relative to the inner phase.

The inner phase of the inventive matrix layer, which matrix layer is made up of two phases, contains at least 25%-wt, preferably at least 50%-wt, and especially preferred more than 70%-wt, of oxybutynin, possibly in combination with desethyloxybutynin.

As binding or thickening agents having the above-described advantages, polymers from the group of the acrylate copolymers and the methacrylate copolymers, preferably basic polymers, e.g. (meth)acrylate copolymers containing amino groups, are particularly suitable. With particular preference, a poly(meth)acrylate copolymer of neutral methacrylic acid esters and dimethylaminoethyl methacrylate is used; such a copolymer is sold under the designation of Eudragit E by the firm of Röhm Pharma.

Furthermore, neutral (meth)acrylate copolymers, for instance a copolymerisate based on methacrylic acid methyl ester and methacrylic acid butyl ester (e.g. Plastoid B; manufacturer: Röhm Pharma), or carboxyl group-free polyacrylate pressure sensitive adhesives (e.g. Durotak 387-2516; by the firm of National Starch) are also particularly suitable as binding or thickening agents. Finally it is also possible that two or more of the polymers mentioned be present in the inner phase as a combination or mixture.

In principle, when selecting the binder polymer(s), one has to make sure that in the formulation of the recipe a stable dispersion or emulsion with small droplet-sizes of the active substance-containing inner phase is obtained. This is favoured by low interfacial energies between the polymers of the inner and the outer phase.

The outer, pressure sensitive adhesive phase (3) is preferably comprised of pure hydrocarbon polymers or/and of silicone polymers. As hydrocarbon polymers, polyisobutylene, polyisoprene, polybutene as well as block copolymers of the styrene-isoprene-styrene and styrene-butadiene-styrene types can be used, for example. To optimize the pressure sensitive adhesive properties, tackifiers from the group of pressure-sensitive adhesive or soft resins can be added.

As an alternative, the outer phase can be prepared on the basis of pressure-sensitive adhesive silicone polymers; especially preferred are amine-resistant polydimethyl siloxanes.

The invention further comprises such embodiments wherein the outer phase contains a combination of at least two different polymer types.

The outer phase has pressure sensitive adhesive properties and serves to anchor the system on the skin; in addition, it has a solubility for the active substance which is as low as possible in order not to impede the release of the active substance. Polymers from the group of the pure hydrocarbons or silicones stand out for their especially low solubility for the active substance oxybutynin base.

In a preferred embodiment, it is provided that the outer phase consists essentially of a mixture of at least two different polyisobutylenes, which possess at least two different molecular weights. Furthermore, in the case of silicone pressure sensitive adhesives being used there is provided a preferred embodiment wherein the outer phase consists substantially of a mixture of at least two different silicone pressure sensitive adhesives which possess at least two different initial tackinesses.

Especially preferred are those embodiments of the inventive TTS wherein the active substance-containing matrix layer(s) do not contain any enhancer substances, so that the risk of skin irritations occurring can be reduced or excluded. Such oxybutynin-containing TTS are substantially free of enhancer substances, i.e. the content of such substances amounts to less than 0.1%-wt, relative to the matrix layer.

Usually the inventive TTS are attached by means of the pressure sensitive adhesive properties of the outer phase. If need be the system can also be provided with an active substance-free pressure-sensitive adhesive overlying patch for better fixation on the skin; suitable possibilities for this purpose are known to those skilled in the art of TTS. It can further be of advantage if between the skin-facing release side of the matrix layer and the detachable protective layer there is arranged a further layer controlling the delivery of the active substance or/and improving anchorage on the skin, for example a membrane controlling the active substance release. Means and methods suitable for this purpose are known to those skilled in the art.

As active substance-impermeable backing layer (1) covering the active substance matrix on the side averted from the skin, polyester films, which stand out for their especially great strength, are particularly suitable, but aside from these also almost any other plastics films well tolerated by the skin, such as, for example, polyvinyl chloride, ethylene vinyl acetate, vinyl acetate, polyethylene, polypropylene, polyethylene terephthalate, cellulose derivatives, and many more. Preferably, the films utilized are water vapour-impermeable.

In the individual case, the backing layer can be provided with an additional coat, e.g. by vapour-deposition of metals or other diffusion-blocking additives such as silicon dioxide, aluminium oxide or similar substances known to those skilled in the art.

The same materials can be used for the detachable protective film (4) as for the backing layer, provided that it is made detachable by a suitable surface treatment, for example siliconization. Other detachable protective layers, such as polytetrafluoroethylene-treated paper, cellophane, polyvinyl chloride or similar materials can, however, be utilized as well.

In the manufacture of the inventive TTS, the polymers of the inner, respectively the outer, phase are dissolved in a solvent, with oxybutynin, possibly in combination with desethyloxybutynin, additionally being admixed to the inner phase. Subsequently, the polymer solutions of the inner, respectively the outer, phase are mixed with one another while stirring, so that a stable emulsion is produced. The emulsion thus-obtained is coated onto a carrier film and dried. In this connection low-molecular hydrocarbons (e.g. n-hexane, cyclohexane, n-heptane, n-octane) are preferably used as solvent for the polymers of the outer phase, and short-chain alcohols, especially preferred ethanol or isopropanol, are preferably used as solvent for the polymers of the inner phase. These conditions yield especially stable emulsions. Mixtures of the solvents mentioned can also be used, e.g. mixtures of the above-mentioned alcohols with ethyl acetate or other acetic acid alkyl esters.

### EXAMPLES

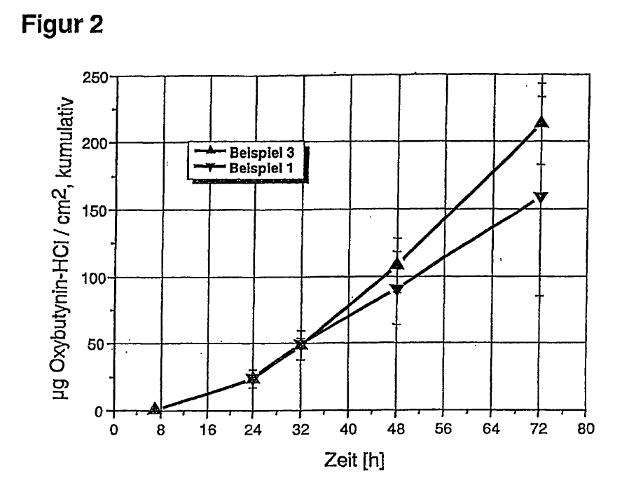

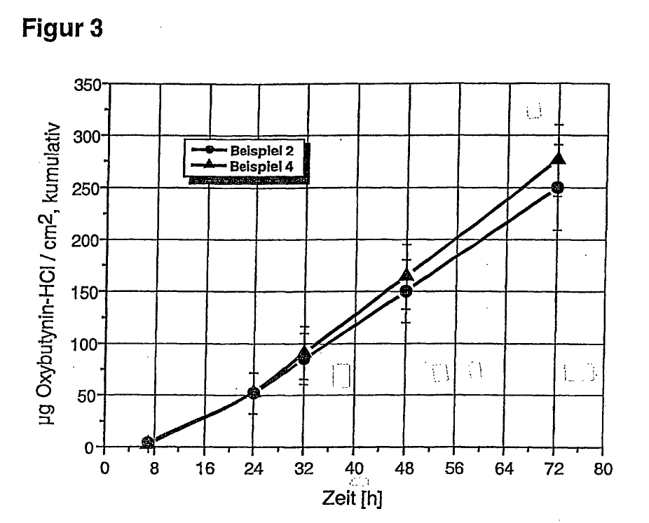

The preparation of the inventive TTS, respectively of the matrix layers contained therein, will be described by means of the following example formulations; in addition thereto the active substance delivery rates experimentally obtained with these formulations are illustrated in FIGS. 2 and 3.

Example Formulations:

Oxybutynin base was isolated from oxybutynin hydrochloride (from the firm of Denk Feinchemie). To this end, the aqueous solution of the hydrochloride was adjusted to a pH value of 10-11, and the free base extracted with diethyl ether. The ether phase was dried over sodium sulphate, and subsequently narrowed down to constancy of weight in the nitrogen stream.

The exemplary recipes mentioned in Table 1 were processed as solutions in organic solvents. The raw materials Oppanol B10 and B100 were dissolved in suitable amounts of petrol, Bio PSA 4301 was used in the form as supplied by Dow Corning as solution in n-heptane.

Eudragit E 100 was used as solution in ethanol, Plastoid B in ethanol/ethyl acetate 1:1 (m/m) was used in ethanol/ethyl acetate 1:1 (m/m), and Durotak 387-2516 was used in the form as supplied by the manufacturer, National Starch.

Oppanol B10 and B100 are polyisobutylenes (by the firm of BASF), Bio PSA 4301 is a silicone-based pressure sensitive adhesive. Oppanol, respectively Bio PSA, forms the outer phase of the matrix layer.

The values in Table 1 refer to the respective portions in %-wt, relative to the weight of the dried matrix layer.

TABLE 1

| | Example 1 | Example 2 | Example 3 | Example 4 |
|---|---|---|---|---|
| Oxybutynin | 15 | 15 | 15 | 15 |
| Plastoid B | 15 | — | — | — |
| Eudragit E | — | 15 | — | 15 |
| Durotak | — | — | 15 | — |
| 387-2516 | | | | |
| Oppanol B10 | 52.5 | 52.5 | 52.5 | — |
| Oppanol | 17.5 | 17.5 | 17.5 | — |
| B100 | | | | |
| Bio PSA | — | — | — | 70 |
| 4301 | | | | |
| Total | 100 | 100 | 100 | 100 |
| Weight per | 85.0 g/m² | 78.0 g/m² | 80.5 g/m² | 85.5 g/m² |
| unit area | | | | |

The adhesive emulsions obtained after thorough stirring with a blade stirrer were coated on siliconized polyester film (PET 100 μm) and dried for 10 min. at the room air, as well as 10 min at 80° C. in a drawing-off air cabinet drier. The resultant films had the weights per unit area listed in Table 1, which are almost identical.

The studies on the permeation of oxybutynin were performed in modified Franz cells on excised human skin at 32° C. As acceptor liquid, an aqueous buffer solution pH 5.5 was used. All indications are based on n=3 skin specimens. The results summarized in FIGS. 2 and 3 each originate from skin specimens of the same skin donor. Each table represents the skin permeation (cumulative) of oxybutynin, calculated as oxybutynin hydrochloride.

The formulations according to the invention in each case yield absorption rates which make a transdermal therapy with oxybutynin at patch sizes of no more than 30 cm² appear possible.

Especially Examples 2 and 4 show short lag times until a constant active substance release through the skin is achieved in vitro.

In these examples, flux rates of up to 4 μg/cm²×h−1 were obtained in steady state.

Thus, it could be shown that with the inventive oxybutynin-containing TTS, sufficient active substance release rates can be obtained without the necessity of adding any enhancer substances.

1. Transdermal therapeutic system (TTS) for administering the active substance oxybutynin, comprising a substantially water vapour-impermeable backing layer (1), at least one pressure-sensitive adhesive matrix layer (2, 3) attached thereto, and a detachable protective film (4), characterized in that the said matrix layer comprises two phases which are immiscible with each other, namely an inner and an outer phase, with the said inner phase (2) containing the active substance oxybutynin base or oxybutynin hydrochloride and being dispersed in the form of droplets in the outer phase (3), and the said outer phase being a pressure sensitive adhesive prepared on the basis of hydrocarbon polymers or/and silicone polymers.

2. TTS according to claim 1, characterized in that the inner phase additionally contains desethyloxybutynin.

3. TTS according to claim 2, characterized in that oxybutynin and desethyloxybutynin are contained in a weight ratio of 1:10 to 10:1.

4. TTS according to any one of claims 1 to 3, characterized in that at least 90%-wt. of oxybutynin and, if contained, also desethyloxybutynin, is present as (S)-enantiomer.

5. TTS according to one of claims 1 to 4, characterized in that at least 50%-wt., preferably 90-100%, of the active substance(s) is dissolved.

6. TTS according to one or more of the preceding claims, characterized in that the inner phase (2) contains an addition of binding agent(s) or thickening agent(s).

7. TTS according to one or more of claims 1 to 6 characterized in that the inner phase (2) contains at least one polymer as binding or thickening agent, said polymer preferably being selected from the group of the acrylate and methacrylate copolymers.

8. TTS according to claim 7, characterized in that the inner phase (2) contains at least one basic polymer, preferably a (meth)acrylate copolymer with a content of amino groups, especially preferred a poly(meth)acrylate copolymer of neutral methacrylic acid esters and dimethylaminoethyl methacrylate.

9. TTS according to claim 7 or 8, characterized in that the inner phase (2) contains at least one neutral (meth)acrylate copolymer, preferably a copolymerisate based on methacrylic acid methyl ester and methacrylic acid butyl ester, or a carboxyl group-free pressure-sensitive polyacrylate adhesive.

10. TTS according to any one of claims 1 to 6 characterized in that the weight percentage of the inner phase (2), relative to the pressure sensitive adhesive layer, amounts to at least 15%, preferably at least 25%.

11. TTS according to one or more of the preceding claims, characterized in that the percentage of the active substance or active substances, relative to the inner phase (2), amounts to at least 25%-wt, preferably at least 50%-wt, and particularly preferred more than 70%-wt.

12. TTS according to one or more of the preceding claims, characterized in that the outer phase (3) substantially consists of a mixture of at least two different polyisobutylenes which have at least two different molecular weights.

13. TTS according to one or more of claims 1 to 11, characterized in that the outer phase (3) substantially consists of a mixture of at least two different silicone pressure-sensitive adhesives which have at least two different initial tackinesses.

14. TTS according to one or more of the preceding claims, characterized in that the active substance-containing pressure sensitive adhesive layer contains at least 5%-wt., preferably 10 to 25%-wt. of oxybutynin base or oxybutynin hydrochloride, optionally in combination with desethyloxybutynin.

15. TTS according to one or more of the preceding claims, characterized in that the active substance-containing matrix layer(s) does/do not contain any additives promoting skin permeation (enhancers).

16. TTS according to one or more of the preceding claims, characterized in that the skin-facing side of the matrix layer is provided with a layer controlling the release of the active substance and/or a layer improving anchorage on the skin.

17. TTS according to one or more of the preceding claims, characterized in that it is produced on the basis of polymer solutions.

18. Process for producing an oxybutynin-containing matrix layer of a TTS according to one or more of the preceding claims, which process is based on the use of polymer solutions, characterized in that
a) the polymer(s) of the inner (2), respectively the outer (3), phase is/are present in dissolved form or are dissolved in a solvent, with low-molecular hydrocarbons preferably being used as the solvent for the polymers of the outer phase, and short-chain alcohols, especially preferred ethanol or isopropanol, or ethyl acetate or mixtures of the aforementioned solvents, preferably being used as the solvent for the polymers of the inner phase;
b) the active substance oxybutynin base or oxybutynin hydrochloride, optionally in combination with desethyloxybutynin is admixed to the polymer solution of the inner phase (2);
c) the polymer solutions of the inner, respectively the outer, phase are mixed with each other by stirring, so that a stable emulsion is produced;
d) the emulsion thus obtained is coated on a carrier film and dried.
